(19) United States
(12) Patent Application Publication
(10) Pub. No.: US 2009/0269445 A1
(43) Pub. Date: Oct. 29, 2009

(54) METHOD AND MICROCAPSULES FOR IMPROVING ORGANOLEPTIC PROPERTIES

(76) Inventors: Patricia Borgstedt, Annandale, NJ (US); Gregory Dardelle, Jonzier Epagny (FR); Sebastien Gouin, Lausanne (CH); Howard Munt, Lawrenceville, NJ (US); Laetitia Nicolas, St-Julien-En-Genevois (FR); Anandaraman Subramaniam, East Windsor, NJ (US); Ki Um, Cranbury, NJ (US)

Correspondence Address: WINSTON & STRAWN LLP;PATENT DEPARTMENT, 1700 K STREET, N.W., WASHINGTON, DC, 20006, US

(21) Appl. No.: 12/304,449

(22) PCT Filed: Jun. 5, 2007
(86) PCT No.: PCT/IB2007/052093

§ 371 Date: Dec. 11, 2008

Related U.S. Application Data
(60) Provisional application No. 60/813,947, filed on Jun. 14, 2006.

(51) Int. Cl. A23L 1/22; B01J 13/08; A23D 9/00; B01J 13/14; A23K 1/16

(52) U.S. Cl. 426/92; 426/534; 426/641; 426/276; 426/652

(57) ABSTRACT

The present invention relates to microcapsules obtained by a coacervation process. The microcapsules encapsulate a heat-treated animal fat and surprisingly increase the mouthfeel properties and juiciness of food product such as meat, dog-food and animal feeds.

### TECHNICAL FIELD

The present invention relates to microcapsules obtained by a coacervation process, in which a heat-treated composition comprising animal fats is encapsulated. The invention further relates to a method for preparing the microcapsules, a food product comprising the microcapsules, and a method for improving organoleptic properties of food.

### BACKGROUND OF THE INVENTION AND PROBLEMS TO BE SOLVED

A constant objective of the flavour industry is to make eating and drinking a better experience such that the addition of a well-defined quantity of a balanced flavour composition to a food product can provide a hedonic experience and improve the overall value of the food product. To achieve this aim, efforts are constantly undertaken to find still new flavour molecules providing new flavour experiences, to create new flavour compositions, to isolate flavours from natural raw materials and yet to find new ways of synthesising flavour compounds.

Generally, encapsulation systems are used to make sure that a given flavour provides its impact only at the predetermined place. Accordingly, the habitual goals of encapsulation are to provide a storage-stable, transportable, and easily processable form of flavours, which still enables release preferably during the moment of consumption and also shortly before that.

The use of encapsulation for flavours is well described. For instance, U.S. Pat. No. 5,759,599 discloses a method for flavouring food, whereby flavours are encapsulated by coacervation, and added to a food material, such as meat or fish, which is subsequently cooked. Other documents reporting the encapsulation of flavours by coacervation are WO 89/04714, U.S. Pat. No. 5,952,007 and U.S. Pat. No. 6,592,916.

In the above-mentioned prior art, particularly in U.S. Pat. No. 5,759,599, the aim of the described invention is to impart a flavor to the meat by injecting into it flavors which are protected from deterioration via encapsulation within a coacervate type system. There are cases however where, instead of flavour, one desires to impart to the end product other characteristics such as mouthfeel and/or juiciness.

It is thus an objective of the present invention to improve organoleptic properties of food in other ways than by addition of flavours. It would, of course be advantageous to improve the organoleptic properties of food by the aid of easily available, inexpensive materials.

More specifically, it is an objective of the present invention to improve the organoleptic properties and mouthfeel of meat, fish and seafood in general. Depending on the way of preparation, meat may get dry during cooking and lose its initial tenderness. It is then generally perceived as hard to chew and tasteless, making its consumption less desirable. It is thus a particular objective of the present invention to increase the juiciness of meat during consumption.

It is a further objective of the present invention to make use of and to improve the value, taste, mouthfeel and overall organoleptic properties of materials of the meat industry, including waste-materials. It is thus an objective to transform tallow, a material that is not known to have any favourable sapidity, to a material that provides added benefits to a food product in terms of overall organoleptic properties.

### SUMMARY OF THE INVENTION

Remarkably, the present inventors found that by subjecting fat-based materials to process involving a heat-treatment, by encapsulating the fat in microcapsules and by adding the encapsulated fat to meat, fish and/or seafood products, the overall organoleptic properties of the food could be distinctly improved. Surprisingly, consumers appreciated the meat products comprising the microcapsules due to increased juiciness and/or mouthfeel.

Accordingly, in a first aspect, the present invention provides microcapsules comprising:
a capsule wall, the capsule wall comprising coacervated, cross-linked colloid protein material, and, optionally, non-protein colloids; and,
an encapsulated material, the encapsulate material comprising
   a heat-treated composition comprising 30-100 wt % of an animal fat; and,
   optionally, 0-10 wt % of added flavours.

In a second aspect, the present invention provides a food product comprising the microcapsules.

In a third aspect, the present invention provides a method for improving the organoleptic properties of a food, the method comprising the step of adding 0.2-5 wt % of the microcapsules of the invention to the food.

In a fourth aspect, the present invention provides a method for improving the juiciness of a meat- and/or fish-based food, the method comprising the step of adding the microcapsules of the invention to the meat- and/or fish-based food.

In a fifth aspect, the present invention provides a method of preparing microcapsules by coacervation, the method comprising the steps of:
heat treating a composition comprising 30-100 wt % of an animal fat;
optionally, adding 0.10 wt % of flavours to the composition;
preparing solution of a protein and, optionally, a non-protein colloid in water, and, suspending or emulsifying particles and/or droplets of the composition in the solution;
forming a colloid wall comprising the protein around the droplets and/or particles of the composition;
cross-linking the colloid wall.

In a sixth aspect, the present invention provides a method for improving the organoleptic properties of animal fat comprising the steps of:
preparing a mixture comprising the animal fat, water, amino acids and sugars;
heat treating the composition at 90-150° C. for 0.5 to 3 hours; and,
optionally, adding one or more hydrophobic, volatile flavours, the added volatile flavours providing 0-10 wt % of the hydrophobic ingredients of the composition.

The term “comprise” or “comprising”, for the purpose of the present invention is intended to mean “including”. It is not intended to mean, “consisting only of”.

The present invention provides a number of unexpected advantages. Firstly, consumers preferred food according to the invention to food which was only flavoured traditionally. It is also an advantage that the encapsulation of animal fats in the microcapsules of the invention, comprising a coacervated wall, enables the injection of ingredients that could otherwise not be injected at room or working temperatures, due to the properties of at these ingredients in there non-encapsulated form at these temperatures.

In addition, the present invention provides a new use for fat based materials, including waste materials, such as tallow. By transforming the waste material in a process involving a heat-treatment, it becomes useful for improving organoleptic properties of food, for example for increasing the juiciness of meat.

### DETAILED DESCRIPTION OF THE PREFERRED EMBODIMENTS

The present invention provides micro-capsules encapsulating a heat-treated animal fat. The heat-treatment is useful to improve the organoleptic quality of the animal fat, which may otherwise be discarded as a waste material.

Accordingly, animal fat having an improved organoleptic quality is obtained in a process comprising a heat treatment. This step may be referred to as a reaction-process, during which ingredients of a mixture interact and may thus modify the organoleptic properties of the animal fat.

The animal fat may be any fat of animal origin, including cattle, deer, sheep, swine, camel, birds, fishes, molluscs, for example. According to a preferred embodiment, the animal fat comprises fat selected from the group of beef fat, pork fat, chicken fat, fat from sheep, optionally hydrolysed fish fat and combinations thereof. According to an embodiment, the animal fat comprises 50-100 wt % of saturated fatty acids. Preferably, the animal fat is tallow, more preferably it is beef tallow.

According to a preferred embodiment, the animal fat has a melting temperature in the range of 15-25° C.

In a first step, a mixture comprising water, amino acids, sugars, the original animal fat, and, optionally, other lipids, is prepared. Typically, this mixture comprises:
50-95 wt % of lipids, including the animal fat;
1.5-10 wt % of water;
0.3-5 wt % of sugars;
0.01-1 wt % of amino acids.

Examples of sugars that can be used for the heat-treatment are sucrose, xylose, glucose, fructose, ribose, maltose, lactose, and so forth. Any of these sugars may be used alone or in the form of a combination comprising two or more of the before-mentioned sugars, or a combination comprising one of the before-mentioned sugars and optionally one or more other sugars.

Any amino acid may be used in the preparation of the above reaction-mixture to be heat-treated. Accordingly, essential and non-essential amino acids may be added to the reaction mixture. For example, alanine, arginine, asparagine, aspartic acid, cysteine, glutamic acid, glutamine, glycine, histidine, isoleucine, leucine, lysine, methionine, phenylalanine, proline, serine, threonine, tryptophane, tyrosine, valine may be used.

Preferably, one or more amino acids are selected from threonine, serine, lysine, histidine, alanine, glycine, cysteine, glutamine, glutamic acid, proline and arginine.

Further ingredients may be present in the mixture before the heat-treatment, generally in low amounts (<2 wt %), such as yeast, yeast extract, flavour enhancers and flavours.

The animal fat generally provides from 30-100 wt % of the lipids of the mixture, preferably 50-90 wt %, more preferably 55-80 wt %. The animal fat may any animal fat, preferably the animal fats mentioned above, or combinations of such fats.

Other lipids that may be added include oils and fats, and fatty acids, for example. Short chain triglycerides, medium chain triglycerides, as well as short chain fatty acids and medium chain fatty acids may thus also be added. Preferably, fatty acids are selected from monounsaturated, polyunsaturated and saturated fatty acids. Preferably, if present, C₈-C₁₈ fatty acids are preferably added to the mixture.

In general, the animal fat provides the predominant component of all the lipids of the mixture. However, the lipids of the mixture to be heat-treated may comprise, besides the animal fat, fatty acids as mentioned above and/or a vegetable oil and/or fat. Accordingly, other lipids such as fatty acids and/or vegetable may provide 10-70 wt %, preferably 20-60 wt %, more preferably 30-60 wt % of the lipids of the mixture. The vegetable oil and/or fatty acids may be selected from canola oil and/or oleic acid, sunflower oil, for example.

Generally, before the heat-treatment, water-soluble ingredients are first dissolved in heated water (45-75° C.), followed by addition of the lipids including the animal fat.

In a further step, the heat-treatment is conducted by heating the mixture to 80-180° C. for 0.3 to 4 hours. This step preferably takes place in a vessel or kettle that can be tightly locked up and that withstands elevated pressures. Preferably, the heat treatment is conducted at 90-150° C. for 0.5-3 hours, and more preferably at 100-140° C. for 0.75-2.5 hours. Preferably, the temperature is kept in the indicated ranges during the heat treatment, more preferably the temperature is kept at an essentially constant value during the entire heat treatment. A heat treatment, for the purpose of the present invention, thus relates to a treatment at temperatures as indicated in this paragraph.

As indicated above, during the heat treatment, the reaction mixture is preferably exposed to an elevated pressure, preferably in the range of 20-40 psi (1 psi≈0.0689 bar≈6895 Pa), more preferably 25-35 psi. This pressure is present in the interior of the vessel of the heat-treatment.

After the heat treatment, the mixture is preferably cooled to temperatures above the melting point of the lipids, and the water comprising residual ingredients is preferably removed to obtain a hydrophobic composition which may further be employed in the preparation of the microcapsules of the present invention. The hydrophobic composition generally comprises 30-100 wt % of an animal fat.

The hydrophobic composition is preferably further purified by filtering.

If the hydrophobic composition comprising the animal fat is not immediately further processed, it may be stored at below-ambient temperatures, preferably at 0-10° C. until further use is made.

In an optional step, flavours may be added to the hydrophobic composition. Generally, only low quantities of flavours are added to the hydrophobic composition.

Flavours are compounds that, due to their high volatility or vapour pressure, reach olfactory receptors in the nose before and during the eating and drinking. In so doing, flavours influence the odour and flavour of a food product. For the purpose of the present invention, a flavour is a compound that is characterised by a vapour pressure of ≧0.01 Pa at 25° C. Most flavours have a vapour pressure above this value, while lipids, such as animal fats, oleic acid, etc, generally have a vapour pressure lower than that.

For the purpose of the present invention and for the sake of convenience, the vapour pressure is determined by calculation. Accordingly, the method disclosed in “EPI suite”; 2000 U.S. Environmental Protection Agency, is used to determine the concrete value of the vapour pressure of a specific compound or component of the ingredient. This software is freely available and is based on average values of vapour pressures obtained by various methods of different scientists.

Preferably, the animal fat-based hydrophobic composition comprises 0-10 wt %, preferably 0.5-7 wt %, or 1-6 wt % of flavours as defined above.

The hydrophobic composition may be a mixture of two or more separately heat-treated hydrophobic compositions, and/or a mixture of a heat-treated composition with lipids, such as oils and fats, that are not heat treated according to the above scheme. In all these cases, the animal fat preferably provides from 30-100 wt % of the hydrophobic composition.

The present invention relates to microcapsules comprising the composition and to a method of making the microcapsules by a coacervation process. The microcapsules are generally water-insoluble, which improves stability in moist food products.

Any coacervation encapsulation process may be used, such as simple and complex coacervation processes, both of which are well known in the art. In simple coacervation, only protein is used to form a capsule wall as phase separation (i.e. “coacervation”) is taking place. Complex coacervation refers to methods in which a generally oppositely charged non-protein polymer and a protein polymer together form the capsule wall. According to the principles of complex coacervation the method of the present invention provides the optional addition of an oppositely charged non-protein polymer, preferably a polysaccharide, to the colloid solution.

Accordingly, in a preferred method of producing microcapsules according to the present invention, a solution is prepared comprising a protein colloid, and, optionally, a non-protein colloid in water.

Then, the heat-treated hydrophobic composition comprising the animal fat obtained above is suspended or emulsified in the colloid solution, in the form suspended particles or suspended or emulsified droplets. Preferably, the hydrophobic composition is liquid at the time of its addition to the colloid solution so that the size of droplets that are suspended or emulsified can easily be adjusted by agitation, for example by stirring.

Preferably, the average droplet size of the emulsified or suspended hydrophobic composition is adjusted to 150-500 μm, preferably, 250 to 350 μm.

The process of preparing microcapsules comprises, as a further step: forming a colloid wall comprising the protein around the droplets and/or particles of the composition. This step is accomplished by inducing a phase separation, that is, the separation of a colloid-rich phase (the coacervate phase) from the remainder of the aqueous solution, the latter being then a phase that is poor in colloids.

Phase separation may be induced in any way known to the person skilled in the art. Preferably, phase separation is achieved by modifying, preferably lowering, pH to or below the iso-electric point of the protein. If a non-protein polymer, for example a polysaccharide is present, the pH is preferably adjusted so that the positive charges on the proteins are neutralized by the negative charges on the non-protein polymer.

The colloid wall of the microcapsule is formed spontaneously once the step of formation of a coacervate phase is induced.

The method of preparing microcapsules of the present invention preferably comprises a step of cross-linking the colloid wall. Cross-linking may be performed in any way, for example by adding sufficient amounts of formaldehyde and/or glutaraldehyde, or enzymatically. Enzymatic cross-linking is effected with the enzyme Transglutaminase. Generally, the step of cross-linking is allowed to continue for 4-30 hours, preferably 6-20 hours, more preferably 8-15 hours, at 5-26° C., for example.

According to a preferred embodiment, the present invention comprises the step of separating the microcapsules from the solution and, optionally, drying them.

A preferred method of drying is spray-drying. For example, carrier materials, such as carbohydrates, etc., may be added to the microcapsules and residual water of the coacervation encapsulation process. The mixture of carrier, water and microcapsules may then be spray-dried.

An important advantage of the encapsulation of animal fat which may have an elevated melting point, for example above 25° C., as is the case with beef tallow, is that it enables injection into food products. If the animal fat having a melting point above room temperature was not encapsulated, injection into a food would not be possible, because of clogging of the injection needles.

According to a preferred embodiment, the microcapsules have an average particle size in the range of 150 to 500 μm, more preferably in the range of 250 to 350 μm.

The particle size can be measured via any well-established method that allows measurements which are accurate within an experimental error of 5% at the most and preferably below 1%. Suitable well established such method resort to the use of laser diffraction measurements and equipment.

The term “average” refers to an arithmetic mean. The particle size may be determined by inspection with the aid of a microscope. For the purpose of determining the diameter of the microcapsules, the capsule wall is not considered. The reason for this is that the capsule wall is not always completely spherical, but typically is ovoid. The uneven formation of the capsule wall is due to the stirring taking place during the encapsulation, which causes droplets to rotate. In contrast, the encapsulated hydrophobic composition forms a nearly spherical droplet surrounded by the capsule wall, and the average diameter of it can thus easily be determined.

The present inventors surprisingly found that, within the above-indicated size ranges, an increased juiciness and mouthfeel is particularly observed. This is because, if the microcapsules are smaller, they tend not to be crushed during chewing and the composition comprising the animal fat, is not released, whereas if the microcapsules are larger, they often break apart too early, for example during the handling and cooking of the meat. Also, microcapsules with diameters above 500 μm can hardly be injected to meat, as the injection needles become clogged thus adversely affecting their industrial processibility.

Food products in which the microcapsules can be used include, for example, bakery foods, instant foods, refrigerated and frozen foods, and fresh foods. Since the microcapsules are water-insoluble, they may also be added to foods having a high water content or high water activity. The microcapsules may be present in raw materials used for the preparation of the food, or in the coating of foods. For example, in frozen or fresh pizza, an effective quantity of microcapsules is present in the pizza dough. Prior to cooking.

The microcapsules of the invention can also be present in food not designed for human consumption. Such food may be animal feed, livestock feed and/or pet food, for example.

According to a preferred embodiment, the food product comprising the microcapsules is food comprising meat or seafood. Meat, for the purpose of the present invention, encompasses red meat, such as beef, pork, sheep, lamp, wildlife, poultry, such as chicken, turkey, goose and duck. Seafood encompasses fish, crustaceans, molluscs, for example. Preferably, the food of the present invention is meat selected from beef, poultry and pork.

The microcapsules may be added in any suitable way to the food, such as by injection, vacuum tumbling, spraying-on optionally with a carrier material or mixing with the food prior to its preparation by extrusion.

If the microcapsules are to be added to ground meat, they may simply be mixed into it. According to a preferred embodiment, the microcapsules are added to the food by injection. For injection, typical injector devices used to inject marinates into meat may be used. On an industrial level, injectors may be commercially obtained from MEPSCO, West Chicago, US, for example. Another technique for incorporating the microcapsules of the invention into food and in particular meat is by vacuum tumbling.

If the food is a pet-food, the microcapsules may be simply added by mixing with other pet-food ingredients. The pet-food is preferably a dry pet-food (Aw<0.3), which is present in the form of kibbles. In this case, the microcapsules may be added to the dry pet-food by a coating or spraying process, for example.

The microcapsules may be by spraying, to pet-food, particularly to pet-food present in the form of wet chunks.

Alternatively, the microcapsules and pet-food ingredients may be mixed and then extruded to obtain kibbles or wet chunks.

Preferably, the pet-food is a dog-food.

Preferably, the food comprises 0.2-5 wt % of microcapsules. Accordingly, the method for improving organoleptic properties of food comprises the step of adding 0.2-5 wt %, preferably 0.5-3.5 wt %, and most preferably 1-3 wt % of the microcapsules to the food product.

The present invention will now be illustrated in greater detail by way of the following examples, but it should be understood that the invention is not construed as being limited thereto.

### EXAMPLES

### Example 1

### Conditioning of Beef Tallow for Improving Mouthfeel Properties

Beef tallow, obtained from Tyson Inc., USA, was subjected to a conditioning treatment for improvement of organoleptic properties and mouthfeel as indicated below. Ingredients used for the conditioning are indicated in Table 1.

TABLE 1
Ingredients for conditioning

| Parts | Ingredients |
|---|---|
| 10-20 | xylose |
| 200-550 | water |
| 0.1-5 | arginine |
| 10-150 | yeast extract |
| 1-30 | cystein |
| 20-140 | glucose |
| 50-110 | yeast |
| 4000-6000 | beef tallow |
| 75-300 | myristic acid |
| 400-1200 | hexanoic acid |
| 4000-6000 | oleic acid |

The water is added into a jacketed pressure kettle and heated to 60° C. All dry ingredients are added into the kettle and the whole is mixed until they are completely dissolved. Finally, the remaining liquid ingredients (beef tallow, oleic acid) are added, followed again by thorough mixing. The kettle is sealed and heated to 130° C. This temperature is then hold for 1.5 hours. A pressure of about 30 psi (207 kPa) was observed in the interior of the kettle during the reaction. At the end of the reaction, the kettle is cooled to 50-65° C. and the agitation is turned off to allow contents to separate for 6 hours. Thereafter, the bottom aqueous phase is drained until a brown oily liquid is observed. The amount drained is generally less than 5 wt % of the overall batch. The product is further cooled to 40-55° C., before filtering the fat and filling it in suitable containers or pouches and storing under refrigeration.

### Example 2

### Conditioning of Chicken Fat for Improving Organoleptic and Mouthfeel Properties

Chicken fat was subjected to a similar reaction as in Example 1, with the difference that two reactions were independently conducted before mixing the oily products of the respective process in a 1:1 ratio. Tables 2 and 3 indicate the ingredients for the conditioning.

TABLE 2
Ingredients for conditioning, part I

| Parts | Ingredients |
|---|---|
| 10-100 | yeast |
| 30-50 | cysteine |
| 1-10 | glutamic acid |
| 40-80 | xylose |
| 40-80 | glucose |
| 300-700 | water |
| 6000-9000 | chicken fat |
| 1-10 | leucine |
| 100-300 | dried chicken broth |
| 1-10 | decadienal |

For part I, the heat-treatment process is identical to that of Example 1 with the following differences: the water at the beginning is heated to 65° C. before dissolving the dry ingredients. The chicken fat constitutes the liquid ingredients that are added at the end. The heat-treatment was conducted at 120° C. for 45 minutes, followed by cooling to 50° C. with chilled water before stopping the agitation and allowing for phase separation.

TABLE 3
Ingredients for conditioning, part II

| Parts | ingredients |
|---|---|
| 1-10 | xylose |
| 300-600 | water |
| 600-1800 | oleic acid |
| 1-10 | arginine |
| 100-500 | yeast extract |
| 1-10 | methionine |
| 20-50 | cystein |
| 7000-9000 | canola oil |
| 1-20 | dextrose |
| 10-100 | yeast |

The heat-treatment process of part II is identical to that of Example 1 with the following differences: The oleic acid and the canola oil constitute the liquid ingredients that are added last. The heat-treatment was conducted at 105° C. for 45 minutes, followed by cooling to 50° C. with chilled water before stopping the agitation and allowing for phase separation.

The fatty liquids of part I and part II are mixed in a 1:1 weight ratio before storing under refrigeration.

### Example 3

### Conditioning of Pork Fat for Improving Organoleptic and Mouthfeel Properties

Pork fat was subjected to a similar reaction as in Example 1. Ingredients are indicated in Table 4.

TABLE 4
Ingredients for conditioning pork fat

| parts | ingredients |
|---|---|
| 15-45 | xylose |
| 300-600 | water |
| 1-10 | arginin |
| 20-150 | yeast extract |
| 20-75 | cystein |
| 20-50 | dextrose |
| 50-100 | yeast |
| 3000-6000 | oleic acid |
| 3000-6000 | pork lard |

The heat-treatment process is identical to that of Example 1 except that the liquid ingredients added last are constituted by the oleic acid and the pork lard. The heat-treatment was conducted at 125° C. for 120 minutes, followed by cooling to 45° C. with chilled water before stopping the agitation and allowing for phase separation.

### Example 4

### Preparation of Microcapsules Comprising Chicken Fat by Coacervation

Poultry gelatine (supplied by Junca) and gum Arabic (Efficacia®, from CNI) are used as the hydrocolloids. A stock solution of gelatine (solution A) is prepared by mixing 180 g of warm deionised water and 20 g of gelatine in a vessel until it is completely dissolved; the solution is then maintained at 40° C. A stock solution of gum Arabic (solution B) is prepared by mixing 180 g of cold deionised water and 20 g of gum Arabic in a vessel until it is completely dissolved; the solution is then warmed and kept at 40° C.

105.4 g of solution A is mixed with 70.3 g of solution B in a vessel under gentle agitation (the gelatine/gum Arabic ratio is 1.5:1). The pH is adjusted to 4.6 with a 50% w/w aqueous lactic solution.

70.3 g of melted chicken fat (the 1:1 composition prepared in Example 2) is slowly added to the gelatine and gum Arabic mixture and homogenised with a stirrer at 350 RPM during 5 min, so as to reach an average droplet size of 300 μm.

The system is then diluted by the addition of 354.1 g of warm deionised water, which brings the total hydrocolloid concentration to 3.4% w/w. The mixture is finally cooled to 20° C. at a rate of 0.5° C. min⁻¹. The stirring speed is slightly decreased, the pH is adjusted to 4.0 and 0.45 g of a 50% glutaraldehyde aqueous solution is added to the mixture. The solution was stirred slowly for 1.5 hours and cross-linking is allowed to proceed overnight at 20° C.

The mixture was then gently dried by fluidized bed drying (Aeromatic MP1) using an inlet temperature of 80° C., an outlet temperature of 40° C. and an air flow of 70 m³/h. The mixture was atomized using a low-pressure double fluid nozzle (1 mm inner diameter) from the bottom of the perforated bottom plate. The product was further dried to a maximum water content of 8% and then discharged into lined containers.

The microcapsules thus obtained were examined under the microscope and had an average diameter of 300 μm as determined by laser diffraction.

### Example 5

### Preparation of Microcapsules Comprising Beef Tallow by Coacervation

14 g beef gelatin (Knox Gelatine, Inc., Cherry Hill, N.J.) was dissolved in 175 ml water at 40° C. 55 g of organoleptically improved melted beef tallow (Example 1) was emulsified in the gelatin solution by stirring at 40° C. A solution of 9.3 g gum arabic (G-85, MCB Chemicals, Norwood, Ohio) in 135 ml water was added to the emulsion, which was then cooled to 18° C. with continuous stirring for 3 hours. The pH was adjusted to 4.0 with diluted acetic acid and 735 microliters of 50 vol % glutaraldehyde in water was added. The solution was stirred slowly for 1.5 hours and cross-linking is allowed to proceed overnight at 20° C.

The drying steps to obtain microcapsules with a water content of maximally 8 wt % were conducted as indicated in Example 4.

The microcapsules thus obtained have an average diameter of 300 μm.

### Example 6

### Preparation of Microcapsules Comprising Pork Fat by Coacervation

6 parts of acid-processed pigskin gelatin having an isoelectric point of 8.2 and 6 parts of gum arabic were dissolved in 30 parts of warm water at 40° C. Then, 30 parts of the improved melted pork lard (Example 3) was added to the above-described colloid solution under vigorous stirring for emulsification to form an o/w type emulsion. The stirring was discontinued with the size of the oil droplets became 300 to 400 micrometers. 200 parts of warm water at 40° C. was added thereto. A 20% aqueous acetic acid solution was added dropwise thereto, while continuing the stirring, to adjust the pH to 4.4. The colloid wall accumulated around the oil droplets was gelled by cooling from the outside of the vessel while continuing the stirring. 1.3 part of transglutaminase enzyme (180 UI/g) was added. The solution was stirred slowly for 2 hours and cross-linking is allowed to proceed overnight at 20° C.

The drying steps to obtain microcapsules with a water content of maximally 8 wt % was conducted as indicated in Example 4.

The microcapsules thus obtained have an average diameter of 350 μm.

### Example 7

### Preparation of Beef-Steaks Having Increased Juiciness

A marinade was prepared comprising 82.5 wt % of water, 3.5 wt % of sodium chloride, 3 wt % of STPP (sodium tripolyphosphate, Na₅P₃O₁₀) and 11 wt % of the microcapsules of Example 5. In a control marinade, the microcapsules were replaced by water (the control thus containing 93.5 wt % water). Accordingly, salt and phosphate were dissolved in water at high shear, then the microcapsules were added under light stirring for 10 minutes.

Frozen beef meat was thawed for 2 days in the fridge, trimmed, and cut to steaks of 1 inch (2.54 cm) thickness. Thereafter, the marinade was injected into the steaks at 10 wt % marinade and 90 wt % steak with an Auvistick® 130 device having a Rotary Filter. The product was vacuum packed and frozen for 2 weeks.

After two weeks, the steaks were again thawed in the fridge and seared in a frying pan at about 160° F. (76° C.), followed by finishing off at 165° F. (74° C.) in the oven.

The heart of each steak is cut out and used for sensory analysis, in which the following attributes were evaluated by the aid of scales from 1-10: (a) beefy smell, (b) beefy taste, (c) tenderness, (d) mouthfeel, (e) overall acceptance.

Consumers appreciated the steaks comprising the microcapsules and noted in particular the increased juiciness and the mouthfeel of these steaks. The control steaks had less juiciness and mouthfeel and had consequently less overall acceptance than the steaks comprising the microcapsules of the present invention.

### Example 8

### Pet-Food Comprising the Microcapsules

The microcapsules of Example 5 were sprayed onto pet-food “croquettes” of commercial origin with a carrier material at 1%.

The pet-food kibbles so obtained contained 1 wt % of the microcapsules of Example 5. In preference tests with dogs, all dogs preferred the kibbles comprising the microcapsules to the untreated kibbles.

1.-13. (canceled)

14. Microcapsules comprising:
a capsule wall comprising coacervated, cross-linked colloid protein material, and, optionally, non-protein colloids; and
an encapsulated material comprising a heat-treated composition comprising 30-100 wt % of an animal fat; and, optionally, 0-10 wt % of added flavors.

15. The microcapsules of claim 14, having an average diameter in the range of 150 to 500 μm.

16. The microcapsules of claim 14, having an average diameter in the range of 250 to 350 μm and in which the animal fat comprises 50-100 wt % of saturated fatty acids.

17. The microcapsules of claim 14, in which the animal fat has a melting temperature in the range of 15-60° C.

18. The microcapsules of claim 14, in which the animal fat comprises fat selected from the group of beef fat, pork fat, chicken fat, fat from sheep, optionally hydrolysed fish fat, or combinations thereof.

19. A food product comprising an edible material and the microcapsules of claim 14.

20. The food product of claim 19, wherein the edible material includes meat or seafood and is in the form of a pet-food or a feed product.

21. The food product of claim 19, wherein the microcapsules are present in an amount of 0.2-5 wt %.

22. A method for improving the organoleptic properties of a food product by adding 0.2-5 wt % of the microcapsules of claim 14 to the edible material that forms the food product.

23. The method of claim 22, wherein the edible material includes meat or seafood and is in the form of a pet-food or a feed product.

24. A method for improving the juiciness or mouthfeel of a meat- or fish-based food product, which comprises adding the microcapsules of claim 14 to the meat- or fish-based food in an amount sufficient to increase juiciness or mouthfeel of the food product.

25. The method of claim 24, wherein the microcapsules are present in the food product in an amount of 0.2-5 wt %.

26. A method of preparing microcapsules by coacervation, which comprises:
preparing a colloid solution of a protein colloid and, optionally, a non-protein colloid in water; and
suspending or emulsifying particles or droplets of a hydrophobic composition in the solution, the hydrophobic composition comprising 30-100 wt % of a heat-treated animal fat;
forming a colloid wall comprising the protein around the droplets or particles of the composition; and
cross-linking the colloid wall to prepare the microcapsules.
